(19) United States
(12) Patent Application Publication
(10) Pub. No.: US 2005/0159731 A1
(43) Pub. Date: Jul. 21, 2005

(54) Intravascular catheter

(76) Inventor: Don W. Lee, Glendale, CA (US)

Correspondence Address: James E. Brunton, Esquire, Post Office Box 29000, Glendale, CA, 91209-9000, US

(21) Appl. No.: 10/759,668

(22) Filed: Jan. 16, 2004

(51) Int. Cl. A61B 17/00

(52) U.S. Cl. 606/1

(57) ABSTRACT

An intravascular catheter system includes an intravascular catheter that can be easily and safely navigated through severely occluded arteries. The system uniquely includes both an optical fiber for use in providing data for guiding the catheter and a conventional metal guide wire for use in navigating the catheter through the artery passageway. The system further includes optical imaging of the arterial occlusion during guidance of the catheter through the artery passageway. More particularly the system provides a visual indication to the surgeon to determine if the catheter assembly is approaching the arterial wall.

### BACKGROUND OF THE INVENTION

1. Field of the Invention

The present invention relates generally to medical devices such as intravascular catheters. More particularly, the invention concerns an intravascular catheter for use in opening partial and total occlusions of an artery passageway.

2. Discussion of the Prior Art

A number of procedures have been suggested in the past for treating disease conditions involving the narrowing or obstruction of the lumen of an artery. This condition, generally referred to as an occlusion, is found in patients suffering from atherosclerosis. An occlusion can manifest itself in hypertension and can be partial or total. The occlusions can be found at various locations in the arterial system, including the aorta, the coronary arteries, the carotid arteries and the peripheral arteries.

In the past, coronary artery occlusions have traditionally been treated by performing coronary bypass surgery, wherein a segment of the patient's saphenous vein is taken from the patient's leg and is grafted onto the affected artery at points upstream and downstream of the occluded segment. While bypass surgery can provide dramatic relief, it involves dangerous open chest surgery and typically a long period of convalescence.

In recent years less invasive procedures have been adopted for the treatment of arterial abnormalities. These procedures typically involve the use a catheter which is introduced into a major artery through a small arterial opening in the patient's body and is advanced into the area of the stenosis.

Popular prior art minimally invasive procedures include percutaneous transluminal coronary angioplasty, directional coronary atherectomy and stenting. Percutaneous transluminal coronary angioplasty typically involves the use of a balloon to mechanically dilate the stenosis. In carrying out this procedure, a steerable guidewire is introduced into an arterial opening and advanced under fluoroscopic observation into the stenosed artery and past the stenosis. This done, a balloon catheter is advanced over the guidewire until it is positioned across the stenosed area. The balloon is then inflated to separate the stenosed tissue.

A somewhat similar prior art procedure, known as stenting, involves the use of a very small wire framework, known as a stent, which is fitted over an inflatable balloon and is then positioned across the stenosed segment of the artery. When the stent is in the proper position, the balloon is inflated, dilating the stent and forcing it against the artery wall.

It is, of course, apparent that over-the-wire catheters cannot be positioned adjacent the stenosis until the guidewire has been advanced across the stenosed area. In those instances where the artery is the occluded, the surgeon may have greater difficulty in guiding the guide wire through the occluded area. For example, the occlusion may contain complex structures which to divert the steering end of the guidewire. Thus, without some type of guidance system, the guidewire might undesirably impinge on and possibly perforate or otherwise damage the artery wall.

In light of the foregoing, there has been a long-felt need to provide a reliable guidance system for guiding a catheter through the occlusion. One prior art guidance system which has been used in conjunction with coronary catheterization involves biplane fluoroscopy, wherein the surgeon observes two flat, real-time x-ray images acquired from different angles. However, biplane fluoroscopy has been proven to be somewhat costly, unreliable and slow.

Recently, promising optical systems have been disclosed for imaging an occlusion through a specially designed catheter positioned within the artery. One such system is Optical Coherence Tomography (OCT). In this system, a beam of light carried by an optical fiber illuminates the artery interior and light reflected back into the fiber from features inside the artery is correlated with the emitted light to capture the depth as well as the angular separation of those features. The features are displayed graphically in two or three dimensions through the use of a suitably programmed computer. Examples of such processing are given in U.S. Pat. No. 5,459,570 issued to Swanson et al. U.S. Pat. No. 5,459,570 is hereby incorporated by reference as though fully set forth herein.

Another prior art guidance system is disclosed in U.S. Pat. No. 6,010,449 issued to Selmon, et al. This patent discloses an intravascular catheter system that includes a steering apparatus, an imaging member and a therapeutic element within a multilumen catheter shaft. In one embodiment of the intravascular catheter system, a rotatable imaging shaft is disposed within the catheter shaft. The imaging shaft contains an optical fiber, which is connected to external optical instruments. At the distal end of the imaging shaft, the optical fiber conducts light from the instruments to illuminate the environment inside the artery and receives optical radiation returned from the environment. The imaging shaft is turned by an external motor encoder, which also measures the rotation of the shaft. As the imaging shaft rotates, the optical beam sweeps circumferentially about the longitudinal axis of the imaging shaft at a fixed angle from the longitudinal axis of the imaging shaft, illuminating different portions of the environment within the artery. The instruments correlate the emitted and received optical data with the rotational data to display an image of the interior of the artery.

Another promising technology for use in catheter guidance systems is Optical Coherence Reflectometry (OCR). The basic concepts of this technology have been well documented (see for example an article by Mandel L. Wolf entitled Optical Coherence and Quantum Optics published in the Cambridge University Press (1995). In the practice of the OCR technology, a light source is divided into two beams, a reference arm and a sample arm. The light in the reference arm is reflected at a determinable path length. Light in the sample is also reflected or scattered by the material present in the sample. The reflections and backscattered light are combined at an optic coupler, and if the path lengths of the two arms are within the coherence length of the light, the light will recorrelate or interfere with one another. The detector measures the interference intensity. Since the reference path length is known and adjustable, the intensity profile of scattered light from a sample can be determined as a function of the reference arm path length.

U.S. Pat. No. 6,451,009 issued to Dasilva, et al. discloses an optical coherence domain reflectometry (OCDR) guided laser ablation device. The Dasilva, et al. device includes a mulitmode laser ablation fiber that is surrounded by one or more single mode optical fibers that are used to image in the vicinity of the laser ablation area to prevent tissue damage. The laser ablation device is combined with an OCDR unit and with a control unit which initializes the OCDR unit and a high power laser of the ablation device. Data from the OCDR unit is analyzed by the control unit and is used to control the high power laser. The OCDR images up to about 3 mm ahead of the ablation surface to enable a user to see sensitive tissue such as a nerve or artery before damaging it by the laser.

A commercially available, prior art catheter system using the OCR technology is sold by IntraLuminal Therapeutics of Carlsbad, Calif. under the name and style “SAFE-STEER”. The IntraLuminal Therapeutics apparatus comprises an optical guide wire with an optical fiber integrated into it. The apparatus also includes an optical coherence reflectometry system which comprises an optical interferometer, a demodulation computer unit and monitor. In one form of the apparatus a single mode fiber with a polyimide jacket is used for the optics. The proximal portion of the guide wire is made up of commercially available hypodermic tubing that serves as a conduit for the fiber. In operation, the backscattered light is analyzed through the low coherence interferometer producing a signal that is displayed and periodically updated on an OCR monitor. The signal is periodically monitored to determine if the normal arterial wall interface is within the field of view. If the normal arterial wall is detected, a visual indication of a red bar is displayed on a monitor and the relative distance to the arterial wall is shown. If the normal arterial wall is not in the field of view, a green bar is displayed indicating that the guidewire can be advanced.

A form of prior art optical fiber guide wire similar to the “SAFE-STEER” guidewire is illustrated and described in an article entitled “Lasers In Surgery: Advanced Characterization Therapeutics, and Systems XI” (Proceedings of The Society of Photo-Optical Instrumentation Engineers, Volume 4244).

A drawback found in certain of the prior art OCR optical fiber guide wire systems resides in the fact that the optical fiber guide wire tends to be substantially more difficult to navigate through the artery passageway than the catheters embodying more conventional metal guide wires such as are used in stent delivery and like procedures. This drawback is uniquely overcome by the apparatus of the present invention which comprises a catheter system that uniquely includes both an optical fiber for use in expeditiously guiding the catheter and a conventional metal guide wire for use in navigating the catheter through the artery passageway.

Still another commercially available, prior art catheter system using radio frequency technology is sold by IntraLuminal Therapeutics of Carlsbad, Calif. under the name and style “SAFE-CROSS.” The Safe Cross system was developed to effectively cross and recanalize total occlusions and according to the manufacturer, comprises a marriage of the OCR technology and controlled Radio Frequency (RF) energy to facilitate guidance through the occlusion.

The IntraLuminal Therapeutics RF apparatus comprises a 0.14 inch support catheter and a 0.35 inch catheter. The apparatus also includes a console and display, a torquer and an advancing mechanism.

### SUMMARY OF THE INVENTION

An object of the present invention is to provide an intravascular catheter system that can be used in the effective treatment of occluded arteries. More particularly, it is an object of the invention to provide such a system which includes an intravascular catheter that can be easily and safely navigated through severely occluded arteries.

Another object of the invention is to provide a system of the aforementioned character that uniquely includes both an optical fiber for use in providing data for guiding the catheter and a conventional metal guide wire for use in navigating the catheter through the artery passageway.

Another object of the invention to provide an intravascular catheter system as described in the preceding paragraphs that includes optical imaging of the arterial occlusion during guidance of the catheter through the artery passageway. More particularly, the system provides a visual indication to the surgeon to determine if the catheter assembly is approaching the arterial wall.

Another object of the invention is to provide an intravascular system that uses a combination of optical imaging and controlled Radio Frequency energy to facilitate guidance through the occlusion.

Another object of the invention is to provide an intravascular catheter system of the class described which is of a simple construction and is easy to use in a conventional manner.

### BRIEF DESCRIPTION OF THE DRAWINGS

FIG. 1 is a generally perspective view of one form of the intravascular catheter system of the present invention.

FIG. 2 is a greatly enlarged, cross-sectional view of the portion of the system designated as “2” in FIG. 1.

FIG. 3 is a cross-sectional view taken along lines 3-3 of FIG. 2.

FIG. 4 is a generally perspective view of an alternative form of the intravascular catheter system of the present invention.

FIG. 5 is a greatly enlarged, cross-sectional view of the portion of the system designated as “5” in FIG. 1.

FIG. 6 is a cross-sectional view taken along lines 6-6 of FIG. 5.

FIG. 7 is a generally perspective view of still another form of the intravascular catheter system of the present invention.

FIG. 8 is a greatly enlarged, cross-sectional view of the portion of the system designated as “8” in FIG. 7.

FIG. 9 is a cross-sectional view taken along lines 9-9 of FIG. 8.

FIG. 10 is a generally diagrammatic, block diagram view of a prior art optical coherence reflectometry system.

FIG. 11 is a generally diagrammatic, block diagram view of one form of the optical coherence reflectometry system of the present invention.

FIG. 12 is a generally perspective view of still another form of the intravascular catheter system of the present invention.

FIG. 13 is a greatly enlarged, cross-sectional view of the portion of the system designated as “13” in FIG. 12.

FIG. 14 is a cross sectional view taken along lines 14-14 of FIG. 13.

### DESCRIPTION OF THE INVENTION

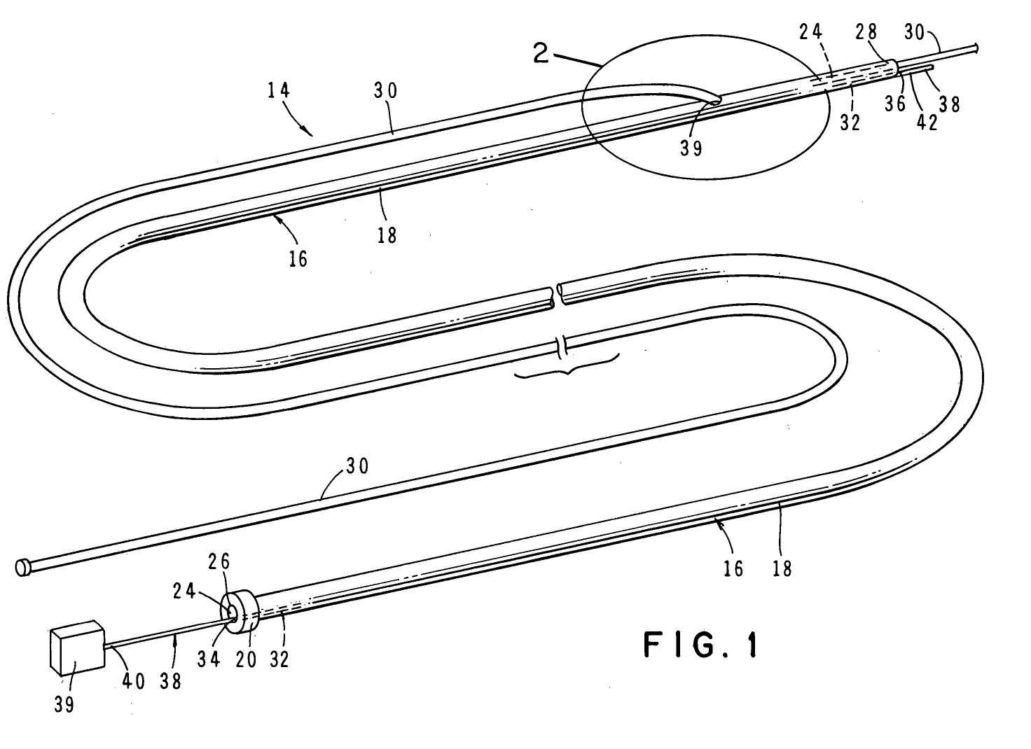

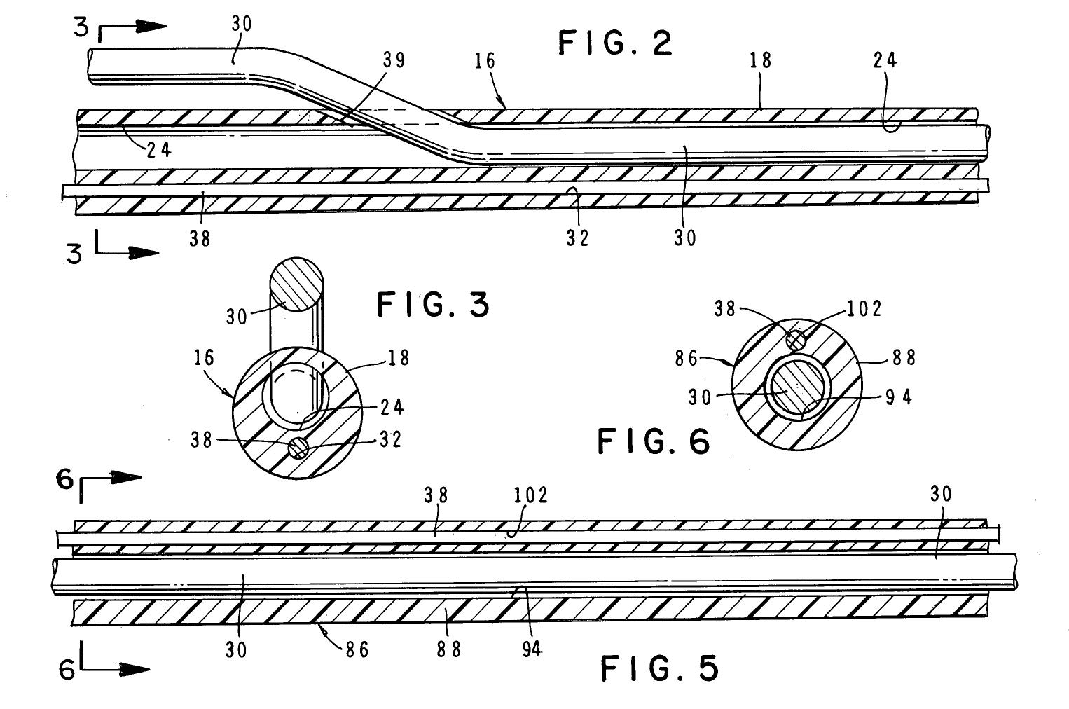

Referring to the drawings and particularly to FIGS. 1 through 3, one form of the intervascular catheter system of the invention is there shown and generally designated by the numeral 14. The catheter system here comprises a catheter 16 having an outer sidewall 18, a proximal end 20 and distal end 22. As been seen by referring to FIGS. 2 and 3, catheter 16 is provided with a first passageway 24 (FIG. 2) having a diameter of about 0.035 inches, a proximal end 26 and a distal end 28. Catheter 16 is preferably formed of a biocompatible and hydrophilic compatible material, such as a lubricous polyimide or polyethylene.

As indicated in FIG. 2, a conventional steerable guide wire 30 is slideably receivable within the first passageway 24 and is movable between first and second positions. While various types of steerable guide wires can be used in the catheter assembly of the invention, guide wire 30 is preferably constructed from a flexible, wire-like metal member having a diameter of on the order of 0.014 inches.

Catheter 16 is also provided with a second passageway 32 that is radially spaced apart from first passageway 24. Second passageway 32 also has a proximal end 34 and a distal end 36. An energy transmission means, shown here as an optical fiber 38, which is carried within second passageway 32 in the manner shown in FIGS. 2 and 3, has a first end 40 and a second end 42, the second end being located adjacent the tip of the catheter and proximate the distal end 36 of second passageway 32. Optical fiber 38, which is of a character well known to those skilled in the art, can be of various sizes, but for the present application preferably has a diameter of on the order of 0.0065 inches. As will be discussed hereinafter, the energy transmission means can also comprise a Radio Frequency (RF) transmitter for transmitting RF energy.

Also comprising a part of the intervascular catheter system of the invention are electronic means which are operably associated with optical fiber 38. These electronic means, which are generally identified in FIG. 1 by the numeral 39, comprise apart of the guidance means of the invention and uniquely provide guidance data to the user of the system to permit to the safe navigation of the catheter through the occlusion. The guidance means along with the optical fiber 38 form a part of the optical coherence reflectometry system (OCR) of the invention the character of which will presently be described.

One form of the method of the invention is carried out using the apparatus shown in FIGS. 1 through 3. This method comprises the steps of first advancing the guide wire 30 through a vessel to a location proximate the occlusion. This done, the catheter 16 is interconnected with the guide wire by inserting the guide wire into a guide wire receiving opening 39 formed in the side wall 18 of the catheter at a location proximate the distal end of the catheter (FIG. 1). Following insertion of the guide wire into the opening 39, the catheter can be controllably advanced over the guide wire to a location where the distal end of the catheter is located proximate the occlusion. As shown in FIG. 1 of the drawings, when the catheter is in position within the occluded vessel of the patient, a substantial portion of the guide wire 30 uniquely resides externally of the catheter. With this novel construction, the guide wire passes through only the distal portion of the central passageway of the catheter in the manner shown in FIG. 1.

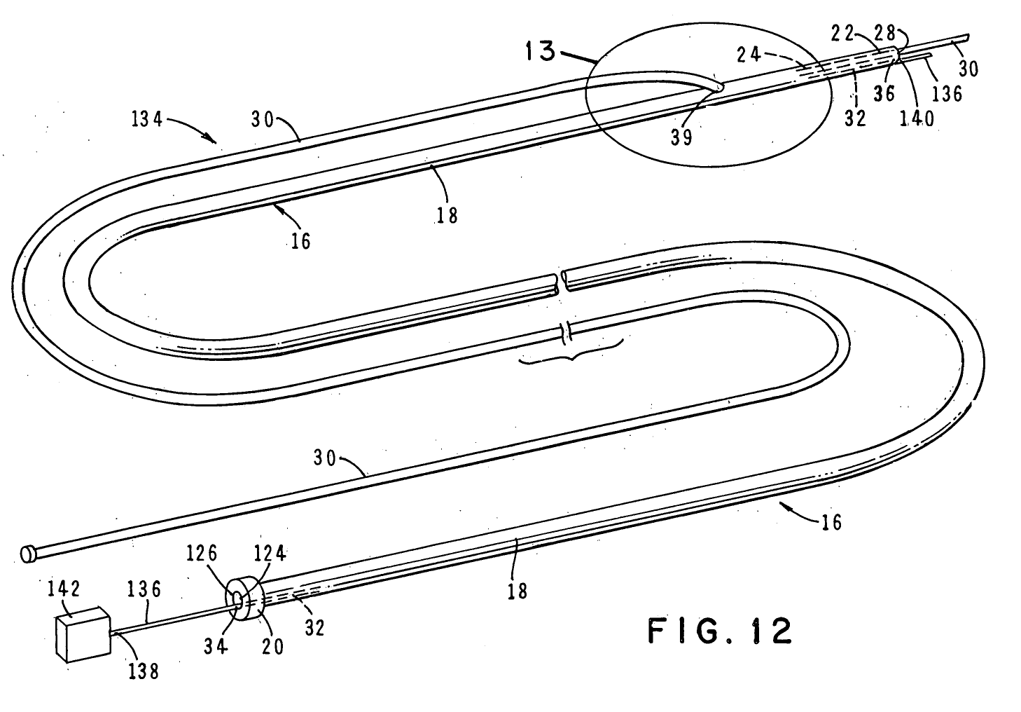

Turning next to FIGS. 10 and 11, it can be seen that the optical coherence reflectometry system of the present form of the invention (FIG. 11) is similar in construction and operation to the prior art optical coherence reflectometry system shown in FIG. 10 which is used for scanning an article. Referring particularly to FIG. 10, the prior art optical coherence reflectometry system there shown can be seen to comprise a low coherence light source 40 that is input into a fiber optic coupler 42 where the light is split and directed into a sample arm 44 and into a reference arm 46, the latter of which provides a variable optical delay. An optical fiber 48 is connected to the sample arm 44 and extends into a device 50, which scans the object 52. Light input into reference arm 46 is reflected back by a reference mirror 54. As shown in FIG. 10, piezoelectric modulator 56 maybe included in reference arm 46. The reflected reference beam from reference arm 46 and a reflected sample beam from sample arm 44 pass back through coupler 42 to detector electronics 58 which processes the signals by techniques well known in the art to produce a backscatter profile (or “image”) that is visually displayed on a suitable display 60. The prior art system shown in FIG. 10 is described in greater detail in U.S. Pat. No. 6,175,669 issued to Colsten et al. which discloses another type of optical fiber guidewire

Turning to FIG. 11, the optical coherence reflectometry system of the apparatus of the present invention comprises a low coherence light source 62 that is input into a conventional fiber optic coupler 64, where the light is split and directed into a sample arm 66 and a reference arm 68. The previously identified optical fiber 38 is connected to sample arm 66 and extends into second passageway 32 of the catheter 16 in the manner shown in FIG. 1. The light in the reference arm 68 is reflected by reflecting means shown here as a mirror 70 at a determinable variable path length when the catheter system is in an initial position within the artery. Right in the sample arm 66 will be reflected or scattered by the material present in the occlusion within which the distal end of the catheter resides. The reflections and backscattered light are combined at a coupler 64 in a manner well understood by those skilled in the art. If the path lengths of the two arms are within the coherence length of the light, the light will re-correlate. A detector 72, which is operably, interconnected with the coupler measures the interference intensity. Detector 72 is also of a character well known in the art. Since the reference path length is known and adjustable, the intensity profile of scattered light from a sample can be determined as a function of the reference arm path length. The scattered light is analyzed by electronic means, which here comprises the electronics 74 and a conventional computer system 76. The cooperative interaction of the electronics and the computer produces a signal tracing that is displayed and periodically updated on a suitable display 78. In a manner well understood by those skilled in the art, the signal tracing is monitored by the computer through a series of algorithms to determine if the arterial wall is within the field of view. If the arterial wall is detected, a visual indication will appear on the display with the catheter assembly in its initial position within the artery if visual indication is not shown on the display, the guidewire can be further advanced a small distance into the inclusion. This done, the catheter is inserted over the guidewire to a position proximate the distal end of the guidewire and the monitor is viewed to verify cautionary visual indication is still not shown on the display. If this is the case, the guidewire can be further inserted a small distance into the occlusion and the catheter then inserted over the guide wire a further distance This procedure can be repeated until a visual indication appears on the display at which point the surgeon must take steps to reroute the steerable guidewire in the direction away from the arterial wall. Unlike the prior art systems which use the optical fiber and its sheath as a guide wire, the apparatus of the present invention, which uniquely embodies a conventional steerable metal guidewire, such as guidewire 38, enables the surgeon to safely and expeditiously navigate through the occlusion with a minimum of a lost time and motion.

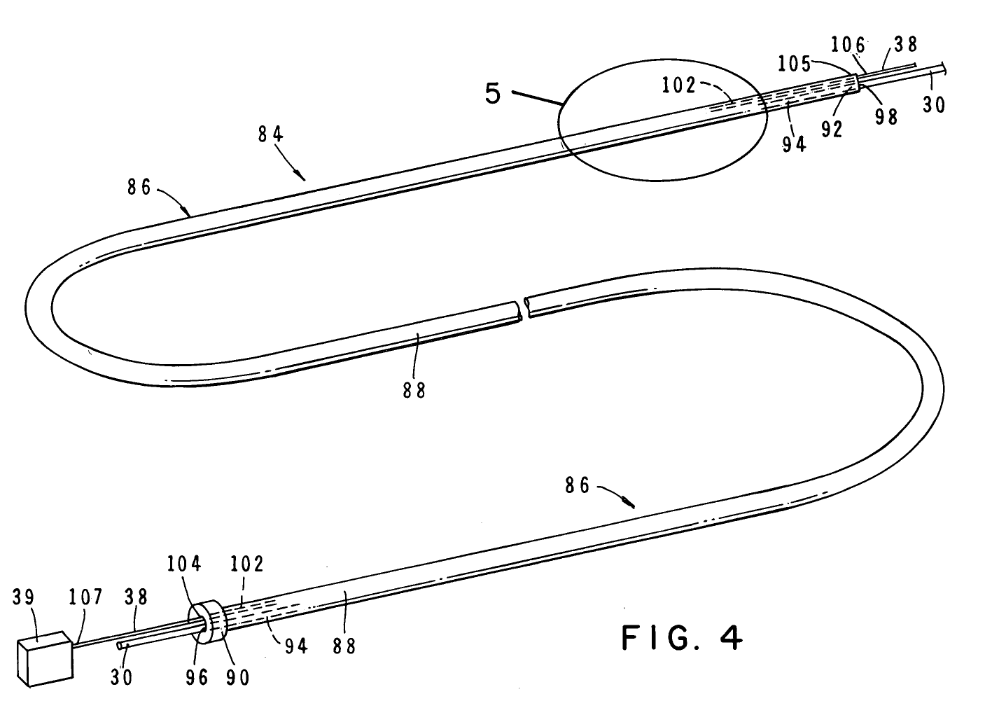

Turning next to FIGS. 4 through 6, an alternate form of the intervascular catheter system as there shown and generally designated by the numeral 84. Catheter system 84 is similar in many respects to that shown in FIGS. 1 through 3 and like numerals are used in FIGS. 4 through 6 to identify like components. As been seen in FIGS. 4 and 5 catheter system 84 comprises a catheter 86 having an outer sidewall 88, a proximal end 90 and distal end 92. Catheter 86 is provided with a first passageway 94 having a proximal end 96 and a distal end 98. Catheter 86, like catheter 14, is preferably formed of a biocompatible and hydrophilic compatible material, such as a lubricous polyimide or polyethylene. The primary difference between catheter 86 and the previously described catheter 14 is that catheter 86 does not include an opening in its side wall for receiving the guide wire and additionally, as shown in FIG. 6, the passageway which receives the guide-wire is axially aligned with the central axis of the catheter.

As indicated in FIGS. 5 and 6, a conventional guide wire 30 is slideably movable within first passageway 94 between a first and second positions. Catheter 86 is also provided with a second passageway 102 which is radially spaced apart from first passageway 94. Second passageway 102 also has a proximal end 104 and a distal end 105. An optical fiber 38, which is carried within second passageway 102 in the manner shown in FIGS. 5 and 6, has a first end 104 and a second end 106, the second end being located proximate the distal end of second passageway 102. Also comprising a part of the intervascular catheter system of this latest form of the invention are instrument means of the character previously described that are operably associated with optical fiber 38 for providing, in the manner previously described, guidance data to the user of the system to permit to the safe navigation of the catheter through the occlusion. The instrument means, along with the optical fiber 38, forms a part of the optical coherence reflectometry system of the invention the character of which is illustrated in FIG. 11 of the drawings. The method of the invention using the alternate embodiment of the invention shown in FIGS. 4 through 6 comprises the steps of first advancing the guidewire 30 through a vessel to a location proximate the occlusion. This done, the catheter 86 is interconnected with the guidewire by inserting the guidewire into the distal end of passageway 94. Following insertion of the guidewire into passageway 94, the catheter is controllably advanced over the guidewire to a location wherein the distal end of the catheter is also proximate the occlusion. The guidewire and the catheter are then incrementally inserted into the occlusion in the manner described in connection with the embodiment of the invention shown in FIGS. 1 through 3 with the surgeon periodically checking the display of the instrument means 39 to make certain that the catheter will not impinge on the artery wall.

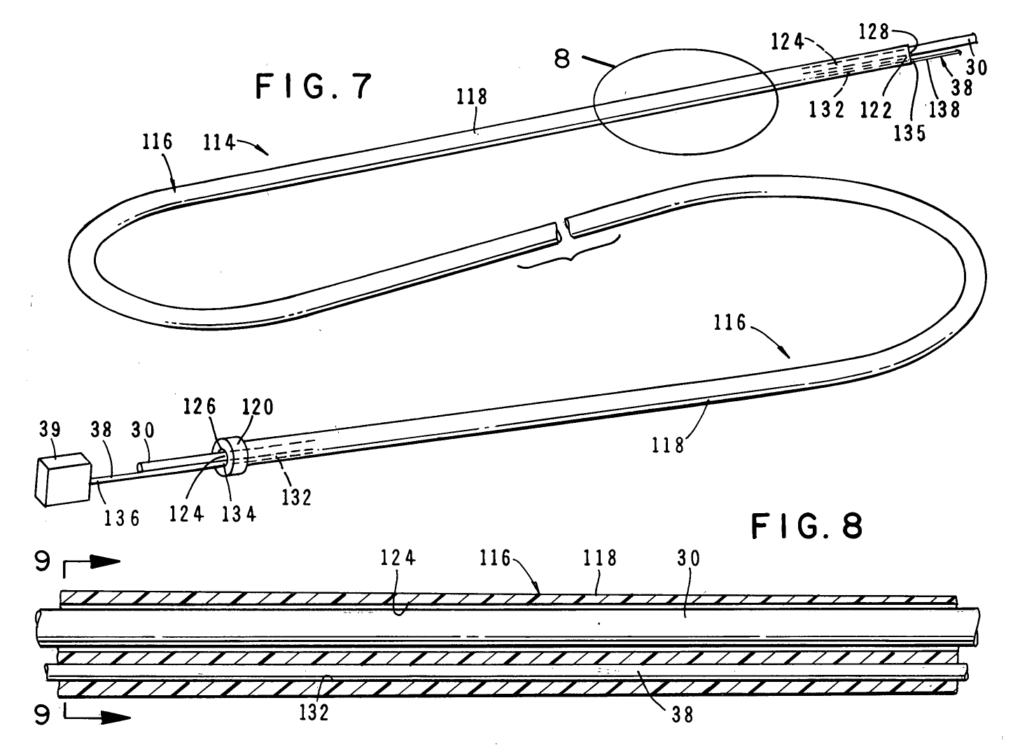

Referring now to FIGS. 7 through 9, still another form of the intervascular catheter system as there shown and generally designated by the numeral 114. Catheter system 114 is similar in many respects to that shown in FIGS. 4 through 6 and like numerals are used in FIGS. 7 through 9 to identify like components. As has been seen in FIGS. 7 and 8 catheter system 114 comprises a catheter 116 having an outer sidewall 118, a proximal end 120 and distal end 122. Catheter 116 is provided with a first passageway 124 having a diameter of approximately 0.035 inches, a proximal and 126 and a distal and 128. Catheter 116, like catheter 84, is preferably formed of a biocompatible and hydrophilic compatible material, such as a lubricous polyimide or polyethylene. The primary difference between catheter 116 and the previously described catheter 84 is that the passageway which receives the guidewire and the passageway that receives the optical fiber are both radially offset from the central axis of the catheter.

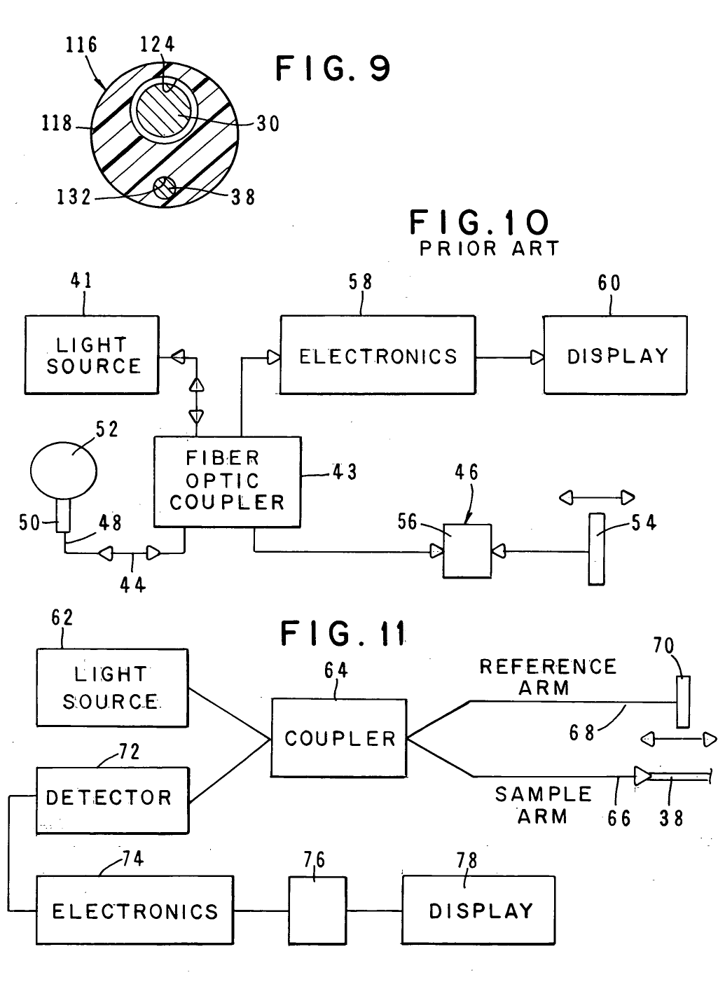

As indicated in FIGS. 8 and 9, a conventional guidewire 30 which has a diameter of about 0.014 inches, is slideably movable within first passageway 124 between a first and second positions. Catheter 116 is also provided with a second passageway 132 which is radially spaced apart from first passageway 124. Second passageway 132 also has a proximal end 134 and a distal end 135. An optical fiber 38, which is carried within second passageway 132 in the manner shown in FIGS. 8 and 9, has a first end 136 and a second end 138, the second end being located proximate the distal end of second passageway 132. Also comprising a part of the intervascular catheter system of this latest form of the invention are instrument means of the character previously described that are operably associated with optical fiber 38 for providing, in the manner previously described, guidance data to the user of the system to permit to the safe navigation of the catheter through the occlusion. The instrument means, along with the optical fiber 38, forms a part of the optical coherence reflectometry system of the invention the character of which is illustrated in FIG. 11 of the drawings. The method of the invention using the alternate embodiment of the invention shown in FIGS. 4 through 6 comprises the steps of first advancing the guidewire 30 through a vessel to a location proximate the occlusion. This done, the catheter 116 is interconnected with the guidewire by inserting the guidewire into the distal end of passageway 124. Following insertion of the guidewire into passageway 124, the catheter is controllably advanced over the guidewire to a location wherein the distal end of the catheter is also proximate the occlusion. The guidewire and the catheter are then incrementally inserted into the occlusion in the manner described in connection with the embodiment of the invention shown in FIGS. 4 through 6 with the surgeon periodically checking the display of the instrument means 39 to make certain that the catheter will not impinge on the artery wall.

Referring next to FIGS. 12, 13 and 14 still another form of the intervascular catheter system of the invention is there shown and generally designated by the numeral 134. This catheter system is similar to that shown in FIGS. 1 through 3 and like numbers are used in FIGS. 12 through 14 to identify like components. The primary difference between system 134 and the earlier described embodiments of the invention resides in the fact that the guidance means for guiding the guide wire comprises a marriage of the previously described OCR technology and controlled radio frequency energy.

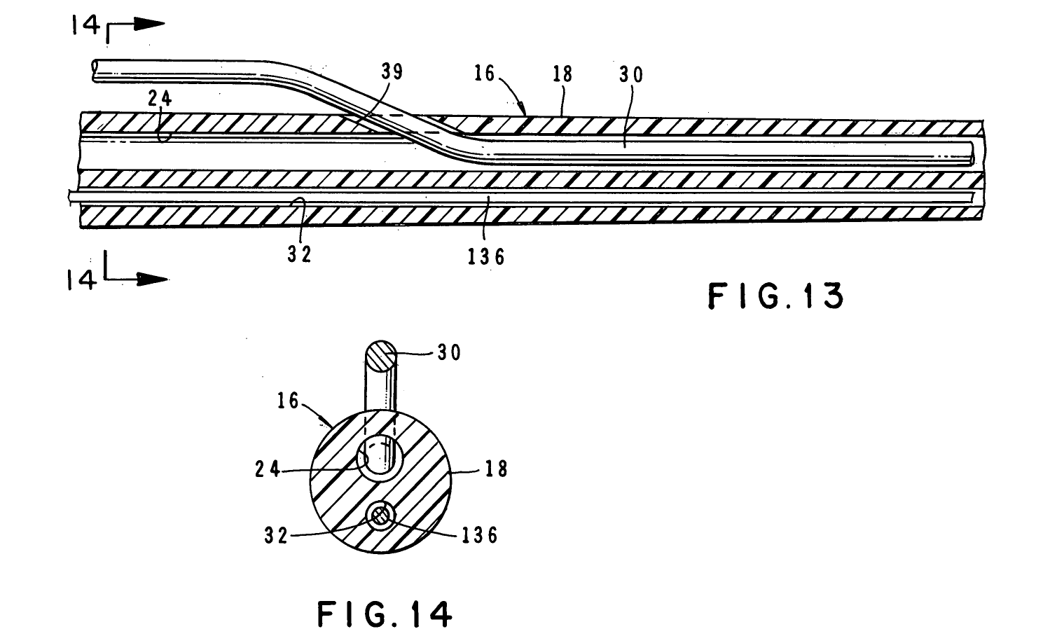

As best seen in FIGS. 12 and 13, system 134 here comprises a catheter 16 of the character previously described having an outer wide wall 18, and proximal end 20 and a distal end 22. As before, catheter 16 is provided with a first passageway 24 (FIG. 13) having a diameter of about 0.035 inches, a proximal end 26 and distal end 28. As indicated in FIG. 13, a conventional steerable guide wire 30 is slideable receivable with the first passageway 24 and is movable between first and second positions.

Catheter 16 is also provided with a second passageway 32 that is radially spaced apart from first passageway 24. Second passageway 32 also has a proximal end 34 and a distal end 36. An energy transmission means, shown here as an energy conduit 136 is carried within second passageway 32. As indicated in FIGS. 13 and 14, conduit 136 has a first end 138 and a second end 140, the second end being located adjacent the tip of the catheter and proximate the distal end 36 of second passageway 32. Energy conduit 136, which is of a character well known to those skilled in the art, can be of various sizes, but for present application preferably has a diameter of on the order of 0.0065 inches. Advantageously, energy conduit 136 can be used to penetrate and cross a total occlusion when such an occlusion is encountered.

Also comprising a part of the intervascular catheter system of the invention are electronic means, which are operably associated with conduit 136. These electronic means, which are generally identified in FIG. 12 by the numeral 142, provide guidance data to the user of the system to permit to the safe navigation of the catheter through the occlusion. A system suitable for use in this latest embodiment of the invention is commercially available from IntraLuminal Therapeutics, Inc. of Carlsbad, Calif. under the name and style “SAFE CROSS.” The details of construction and operation of this RF system are available from this company.

An alternate form of the method of the invention is carried out using the apparatus shown in FIGS. 12, 13 and 14. This method comprises the steps of first advancing the guide wire 30 through a vessel to a location proximate the occlusion. This done, the catheter 16 is interconnected with the guide wire by inserting the guide wire into a guide wire receiving opening 39 formed in the side wall 18 of the catheter at a location proximate the distal end of the catheter (FIG. 12). Following insertion of the guide wire into the opening 39, the catheter can be controllably advanced over the guide wire to a location where the distal end of the catheter is located proximate the occlusion. As shown in FIG. 12 of the drawings, when the catheter is in position within the occluded vessel of the patient, a substantial portion of the guide wire 30 uniquely resides externally of the catheter. With this novel construction, the guide wire passes through only the distal portion of the central passageway of the catheter in the manner shown in FIG. 12.

Guidance of the wire is then accomplished using the guidance means of the invention which here comprises the previously identified “SAFE CROSS” system. The details of the use of this system are available from the previously identified Infraluminal company.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

1. A catheter system comprising:
(a) a guide wire;
(b) a catheter having an outer side wall, a proximal end and distal end and including:
(i) a first passageway through which the guide wire can be slideably moved between a first possession and a second position;
(ii) a second passageway spaced apart from said first passageway, said second passageway having a proximal end and a distal end;
(c) guidance means for guiding travel of said guide wire, said guidance means comprising:
(i) energy transmission means; received within said second passageway, said energy transmission means having a first end and a second end, said second end being located proximate said distal end of said second passageway;
(ii) a source of energy operably associated with said energy transmission means for directing energy toward the said energy transmission means;
(iii) detector means operably associated with said energy transmission means for receiving a signal from said energy transmission means;
(iv) electronic means operably associated with said detector means for analyzing said signal and for generating a signal tracing; and
(v) display means operably associated with said electronic means for displaying said signal tracing.

2. The catheter system as defined in claim 1 in which said outer side wall of said catheter is provided with an opening in communication with said first passageway for receiving said guide wire there through.

3. The catheter system as defined in claim 1 in which said catheter has an axial centerline and in which said first passageway is aligned with said axial centerline.

4. The catheter system as defined in claim 1 in which said catheter has an axial centerline and in which said second passageway is aligned with said axial centerline.

5. The catheter system as defined in claim 1 in which said source of energy comprises a low coherence light source.

6. The catheter system as defined in claim 1 in which said source of energy comprises a radio frequency transmitter.

7. A catheter system comprising:
(a) a guide wire;
(b) a catheter having an outer side wall, a proximal end and distal end and including:
(i) a first passageway through which the guide wire can be slideably moved between a first position and a second position;
(ii) a second passageway spaced apart from said first passageway, said second passageway having a proximal end and a distal end;
(c) an optical fiber received within said second passageway, said optical fiber having a first end and a second end, said second end being located proximate said distal end of said second passageway;
(d) illumination means operably associated with said optical fiber for directing light toward the said optical fiber;
(e) reflecting means disposed proximate said optical fiber for reflecting light from said optical fiber;
(f) a fiber optic coupler operably associated with said optical fiber for receiving light reflected in from said optical fiber and for generating a signal in response thereto;
(g) a detector operably interconnected with said coupler for receiving said signal from said fiber optic coupler;
(h) electronic means operably associated with said detector for analyzing said signal and for generating a signal tracing; and
(i) display means operably associated with said electronic means for displaying said signal tracing.

8. The catheter system as defined in claim 7 in which said outer side wall of said catheter is provided with an opening in communication with said first passageway for receiving said guide wire therethrough.

9. The catheter system as defined in claim 7 in which said catheter has an axial centerline and in which said first passageway is aligned with said axial centerline.

10. The catheter system as defined in claim 7 in which said catheter has an axial centerline and in which said second passageway is aligned with said axial centerline.

11. The catheter system as defined in claim 7 in which said illumination means comprises a low coherence light source.

12. The catheter system as defined in claim 7 in which said reflecting means comprises a mirror.

13. A catheter system comprising:
(a) a guide wire;
(b) a catheter having an outer side wall having an opening therein, a proximal end and distal end and including:
(i) a first passageway through which the guide wire can be slideably moved between a first position and a second position, said first passageway being in communication with said opening in said outer side wall of said catheter;
(ii) a second passageway spaced apart from said first passageway, said second passageway having a proximal end and a distal end;
(c) an optical fiber received within said second passageway, said optical fiber having a first end and a second end, said second end being located proximate said distal end of said second passageway.
(d) illumination means operably associated with said optical fiber for directing light toward the said optical fiber, said illumination means comprising a low coherence light source;
(e) reflecting means disposed proximate said optical fiber for reflecting light from said optical fiber, said reflecting means comprising a mirror;
(f) a fiber optic coupler operably associated with said optical fiber for receiving light reflected in from said optical fiber and for generating a signal in response thereto;
(g) a detector operably interconnected with said coupler for receiving said signal from said fiber optic coupler;
(h) electronic means operably associated with said detector for analyzing said signal and for generating a signal tracing; and
(i) display means operably associated with said electronic means for displaying with signal tracing.

14. The catheter system is defined in claim 13 in which said guidewire comprises a steerable metal guide wire having a diameter of approximately 0.014 inches.

15. The catheter system as defined in claim 13 in which said catheter has an axial center line and in which said first and second passageways are radially offset from said axial centerline.

16. A method for opening occlusions in an artery passageway using a catheter system comprising a steerable guide wire having first and second ends, a catheter having a distal end and a proximal end and a first passageway through which the guidewire can be slideably moved, a second passageway spaced apart from said first passageway, and an optical coherence reflectometry system including an optical fiber received within said passageway, said method comprising the steps of:
(a) inserting the guidewire into the occluded artery to a position where said second end thereof resides within the occlusion;
(b) inserting the first end of the guidewire into the first passageway of the catheter and sliding the catheter over the guidewire to a position where and the distal end of the guidewire resides within the occlusion;
(c) using the optical coherence reflectometry verifying that the catheter is not approaching the wall of the artery;
(d) advancing the second end of the guidewire into the occlusion;
(e) using the optical coherence reflectometry verifying that the catheter is not approaching the wall of the artery; and
(f) further advancing the second end of the guidewire into the occlusion.

17. The method as defined in claim 16, including the further steps of sequentially advancing the second end of the guidewire into the inclusion until the occlusion is opened and as the guidewire is advanced periodically using the optical coherence reflectometry to verify that the catheter is not approaching the wall of the artery.

18. A method for opening occlusions in an artery passageway using a catheter system comprising a steerable guidewire having first and second ends, a catheter having a distal end and a proximal end, a side wall having an opening therein and a first passageway through which the guidewire can be slideably moved, said first passageway being in communication with said opening in said side wall of said catheter, the catheter further having a second passageway spaced apart from said first passageway, and an optical coherence reflectometry system including an optical fiber received within sid second passageway, said method comprising the steps of:
(a) inserting the guidewire into the occluded artery to a position where said second end thereof resides within the occlusion;
(b) inserting the first end of the guidewire into the first passageway of the catheter via the opening of a side wall of the catheter and sliding the catheter over the guidewire to a position where and the distal end of the guidewire resides within the occlusion;
(c) using the optical coherence reflectometry verifying that the catheter is not approaching the wall of the artery;
(d) advancing the second end of the guidewire into the occlusion;
(e) using the optical coherence reflectometry verifying that the catheter is not approaching the wall of the artery; and
(f) further advancing the second end of the guidewire into occlusion.

19. The method as defined in claim 18 including the further steps of sequentially advancing the second end of the guidewire into the inclusion until the occlusion is opening and as the guidewire is advanced periodically using the optical coherence reflectometry to verify that the catheter is not approaching the wall of the artery.

20. The method as defined in claim 19 including the further stop of removing the guidewire and the catheter from the artery.
